(19) United States
(12) Patent Application Publication
(10) Pub. No.: US 2022/0009821 A1
(43) Pub. Date: Jan. 13, 2022

(54) BIOACTIVE PHOSPHATE GLASSES

(71) Applicant: Corning Incorporated, Corning, NY (US)

(72) Inventors: Qiang Fu, Painted Post, NY (US); Lina Ma, Corning, NY (US)

(21) Appl. No.: 17/294,123

(22) PCT Filed: Nov. 13, 2019
(86) PCT No.: PCT/US2019/061077

§ 371 Date: May 14, 2021

Related U.S. Application Data
(60) Provisional application No. 62/769,845, filed on Nov. 20, 2018.

(51) Int. Cl. C03C 3/19; C03C 3/064; C03C 4/00; C03C 12/00

(52) U.S. Cl. CPC C03C 3/19; C03C 3/064; C03C 2203/10; C03C 12/00; C03C 2203/50; C03C 4/0021

(57) ABSTRACT

The disclosure relates to bioactive glasses for use in biomedical applications. In particular, the glasses described herein are phosphate glasses that show fast filling rates of dentin tubules and have advantageous release rates of metal ions, which provide advantages in antibacterial applications and wound healing.

### CROSS-REFERENCE TO RELATED APPLICATIONS

This application claims the benefit of priority of U.S. Provisional Application Ser. No. 62/769,845 filed on Nov. 20, 2018 the content of which are relied upon and incorporated herein by reference in its entirety as if fully set forth below.

### FIELD OF DISCLOSURE

The disclosure relates to bioactive glasses for use in biomedical applications. In particular, the glasses described herein are borate glasses that show fast filling rates of dentin tubules and have advantageous release rates of metal ions, which provide advantages in antibacterial applications and wound healing.

### BACKGROUND

Bioactive glasses are a group of glass and glass ceramic materials that have shown biocompatibility or bioactivity, which has allowed them to be incorporated into human or animal physiology. A number of these materials exist on the market already, such as Bioglass 8625, a soda-lime glass used for encapsulation of implanted devices, and Bioglass 45S5, a bioactive glass composition used in bone repair. However, there continues to be an unmet need for solutions to biomedical problems that novel biocompatible inorganic compositions may help resolve.

### SUMMARY

In an aspect (1), the disclosure provides a glass composition comprising, in wt %: wt %: 0-25 SiO₂, 0-15 B₂O₃, 50-90 P₂O₅, 0-10 Al₂O₃, 0-5 Li₂O, 0-15 Na₂O, 0-15 K₂O, 0-10 MgO, 1-25 CaO, 5-30 MO, 0-15 R₂O, and 70 wt % or greater (P₂O₅+CaO) wherein MO is the sum of MgO, CaO, SrO, and BaO, R₂O is the sum of Na₂O, K₂O, Li₂O, and Rb₂O. In another aspect (2), the disclosure provides the glass composition of aspect (1), wherein the glass composition comprises >0-10 wt % Na₂O. In another aspect (3), the disclosure provides the glass composition of aspect (1), wherein the glass composition comprises 2-8 wt % Na₂O. In an aspect (4), the disclosure provides the glass composition of any of aspects (1)-(3), wherein the glass composition comprises >0-15 wt % B₂O₃. In another aspect (5), the disclosure provides the glass composition of aspect (1), wherein the glass composition comprises 75 wt % or greater (P₂O₅+CaO). In another aspect (6), the disclosure provides the glass composition of aspect (5), wherein the glass composition comprises 80 wt % or greater (P₂O₅+CaO) In an aspect (7), the disclosure provides the glass composition of any of aspects (1)-(6), wherein the glass composition comprises: >0-5 wt % MgO. In an aspect (8), the disclosure provides the glass composition of any of aspects (1)-(7), wherein the glass composition comprises: >0-8 wt % ZnO₂.

In another aspect (9), the disclosure provides the glass composition of any of aspects (1)-(8), wherein the glass composition comprises: 59-70 wt % P₂O₅; 2-15 wt % B₂O₃; and 5-25 wt % CaO. In an aspect (10), the disclosure provides the glass composition of any of aspects (1)-(8), wherein the glass composition comprises: 70-80 wt % P₂O₅; 9-15 wt % CaO; and is essential free of Al₂O₃. In an aspect (11), the disclosure provides the glass composition of any of aspects (1)-(8), wherein the glass composition comprises: 70-80 wt % P₂O₅; 5-15 wt % CaO; and 1-5 wt % Na₂O. In an aspect (12), the disclosure provides the glass composition of any of aspects (1)-(11), wherein the glass composition further comprises: 0-5 wt % ZrO₂. In an aspect (13), the disclosure provides the glass composition of any of aspects (1)-(12), wherein the glass composition is essentially free of or comprises 1 wt % or less of Li₂O. In an aspect (14), the disclosure provides the glass composition of any of aspects (1)-(13), wherein the glass composition is essentially free of or comprises 1 wt % or less of SiO₂.

In another aspect (15), the disclosure provides the glass composition of any of aspects (1)-(14), wherein the glass composition is in the form of powder, particles, beads, particulates, short fibers, long fibers, or woolen meshes.

In another aspect (16), the disclosure provides a method of making the glass composition of any of aspects (1)-(15), the method comprising, mixing the requisite batch oxides to form a mixture; and extracting the mixture to form a glass comprising the composition. In another aspect (17), the disclosure provides the method of aspect (16), wherein the glass is milled to form a plurality of particles, the particles having a particle distribution that is approximately Gaussian. In another aspect (18), the disclosure provides the method of aspect (17), wherein the glass composition is in the form of a plurality of particles, and the particles have an average particle size of from 10 microns to 100 microns.

In an aspect (19), the disclosure provides an oral care composition comprising the glass composition of any of aspects (1)-(15). In an aspect (20), the disclosure provides the composition of aspect (19), wherein the glass composition is in the form of a plurality of particles, the particles having a particle distribution that is approximately Gaussian. In an aspect (21), the disclosure provides the composition of aspect (20), wherein the glass composition is in the form of a plurality of particles, and the particles have an average particle size of from 10 microns to 100 microns. In an aspect (22), the disclosure provides the composition of any of aspects (20) or (21), wherein the composition further comprises glycerol, sodium lauryl sulfate, silicon dioxide, polyethylene glycol, and/or a saccharin salt.

In an aspect (23), the disclosure provides a method for treating an adverse dental condition in the oral cavity, the method comprising contacting the oral care composition of any of aspects (20)-(22) with the oral cavity for a time sufficient to ameliorate the adverse dental condition. In an aspect (24), the disclosure provides the method of aspect (23), wherein the adverse dental condition is tooth decay, bleeding gums, gum disease, gingivitis, dental hypersensitivity, halitosis, oral infection, or periodontal disease.

In an aspect (25), the disclosure provides a treatment composition for wound care management of a biological tissue, the treatment composition comprising the glass composition of any of aspects (1)-(15). In an aspect (26), the disclosure provides the composition of aspect (25), wherein the treatment composition is in the form of an article including the composition, for example, a liquid vehicle or solid support, a wound dressing, a stent, an implant, a bandage, an ointment, a salve for oral or topical application, a dosage form for oral or topical administration.

In an aspect (27), the disclosure provides a method of wound healing, the method comprising contacting at least part of the biological tissue in the wound with the wound healing composition of aspect (25) or aspect (26), for a time sufficient to allow the composition to enhance healing.

### DETAILED DESCRIPTION

In the following description, whenever a group is described as comprising at least one of a group of elements and combinations thereof, it is understood that the group may comprise, consist essentially of, or consist of any number of those elements recited, either individually or in combination with each other. Similarly, whenever a group is described as consisting of at least one of a group of elements or combinations thereof, it is understood that the group may consist of any number of those elements recited, either individually or in combination with each other. Unless otherwise specified, a range of values, when recited, includes both the upper and lower limits of the range as well as any ranges therebetween. As used herein, the indefinite articles “a,” “an,” and the corresponding definite article “the” mean “at least one” or “one or more,” unless otherwise specified. It also is understood that the various features disclosed in the specification and the drawings can be used in any and all combinations.

Where a range of numerical values is recited herein, comprising upper and lower values, unless otherwise stated in specific circumstances, the range is intended to include the endpoints thereof, and all integers and fractions within the range. It is not intended that the scope of the claims be limited to the specific values recited when defining a range. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. Finally, when the term “about” is used in describing a value or an end-point of a range, the disclosure should be understood to include the specific value or end-point referred to. When a numerical value or end-point of a range does not recite “about,” the numerical value or end-point of a range is intended to include two embodiments: one modified by “about,” and one not modified by “about.”

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. It is noted that the terms “substantially” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. Thus, a glass that is “free” or “essentially free” of Al₂O₃ is one in which Al₂O₃ is not actively added or batched into the glass, but may be present in very small amounts as a contaminant (e.g., 500, 400, 300, 200, or 100 parts per million (ppm) or less or).

Herein, glass compositions are expressed in terms of wt % amounts of particular components included therein on an oxide bases unless otherwise indicated. Any component having more than one oxidation state may be present in a glass composition in any oxidation state. However, concentrations of such component are expressed in terms of the oxide in which such component is at its lowest oxidation state unless otherwise indicated.

Unless otherwise specified, all compositions are expressed in terms of weight percent (wt %). Coefficients of thermal expansion (CTE) are expressed in terms of 10⁻/° C., unless otherwise specified. The CTE can be determined, for example, using the procedure described in ASTM E228 “Standard Test Method for Linear Thermal Expansion of Solid Materials with a Push-Rod Dilatometer” or ISO 7991:1987 “Glass—Determination of coefficient of mean linear thermal expansion.” The density in terms of grams/cm³ was measured via the Archimedes method (ASTM C693). Young's modulus, shear modulus, and Poisson's Ratio were measured via the ASTM C623 standard.

### Glass Compositions

Bioactive glasses are a group of glass and glass ceramic materials that have shown biocompatibility or bioactivity, which has allowed them to be incorporated into human or animal physiology. The biocompatibility and in vivo properties of the glass are influenced by the glass composition. In the glass compositions described herein, P₂O₅ serves as the primary glass-forming oxides. Phosphate glasses are generally much less durable than silicate glasses, making them attractive for fast degradation. However, the potential toxicity caused by the degradation and the difficulties in controlling the degradation rate make using these materials a continuing challenge.

In some embodiments, the glass comprises a combination of P₂O₅ and CaO. In some embodiments, the glass further comprises Al₂O₃, B₂O₃, SiO₂, K₂O, and/or Na₂O. For example, embodiments may comprise a glass composition comprising, in wt %: wt %: 0-25 SiO₂, 0-15 B₂O₃, 50-90 P₂O₅, 0-10 Al₂O₃, 0-5 Li₂O, 0-15 Na₂O, 0-15 K₂O, 0-10 MgO, 1-25 CaO, 5-30 MO, 0-15 R₂O, and 70 wt % or greater (P₂O₅+CaO) wherein MO is the sum of MgO, CaO, SrO, and BaO, R₂O is the sum of Na₂O, K₂O, Li₂O, and Rb₂O. In additional embodiments, the glass composition comprises >0-10 wt % Na₂O. In some embodiments, the glass composition comprises 2-8 wt % Na₂O. In some embodiments, the glass composition additionally comprises >0-15 wt % B₂O₃. In some embodiments, the glass composition comprises 75 wt % or greater (P₂O₅+CaO). The phosphate glasses disclosed herein are particularly suitable for biomedical or bioactive applications.

SiO₂, which is an optional oxide component of the embodied glasses, may be included to provide high temperature stability and chemical durability. In some embodiments, the glass can comprise 0-25 wt % SiO₂. In some embodiments, the glass can comprise 10 wt % or less SiO₂. In some embodiments, the glass can comprise 1 wt % or less SiO₂. In some embodiments, the glass is essentially free of SiO₂. In some embodiments, the glass can comprise 0-25 wt %, >0-25 wt %, 1-25 wt %, 5-25 wt %, 10-25 wt %, 0-20 wt %, >0-20 wt %, 1-20 wt %, 5-20 wt %, 10-20 wt %, 0-15 wt %, >0-15 wt %, 1-15 wt %, 5-15 wt %, 10-15 wt %, 0-10 wt %, >0-10 wt %, 1-10 wt %, 5-10 wt %, 0-5 wt %, >0-5 wt %, 1-5 wt %, 0-1 wt %, or >0-1 wt % SiO₂. In some embodiments, the glass is essentially free of SiO₂ or comprises 0, >0, 1, 2, 3, 4, 5, 6, 7, 8, 9, 10, 11, 12, 13, 14, 15, 16, 17, 18, 19, 20, 21, 22, 23, 24, or 25 wt % SiO₂.

Without being bound by theory, in borate glasses, B₂O₃ is the fundamental glass former due to the higher bond strength, lower cation size, small heat of fusion and trivalent nature of B. In these glasses, B³⁺ ions are triangularly or tetrahedrally coordinated by oxygen and corner-bonded in a random configuration. In some embodiments, the glass can comprise 0-15 wt % B₂O₃. In some embodiments, the glass can comprise 5-15 wt % B₂O₃. In some embodiments, the glass can comprise 0-5 wt % B₂O₃. In some embodiments, the glass can comprise from 0-15 wt %, >0-15 wt %, 2-15 wt %, 5-15 wt %, 8-15 wt %, 10-15 wt %, 0-10 wt %, >0-10 wt %, 2-10 wt %, 5-10 wt %, 0-8 wt %, >0-8 wt %, 2-8 wt %, 5-8 wt %, 0-5 wt %, >0-5 wt %, or 2-5 wt % B₂O₃. In some embodiments, the glass can comprise 0, >0, 1, 2, 3, 4, 5, 6, 7, 8, 9, 10, 11, 12, 13, 14, or 15 wt % B₂O₃.

The addition of alumina to borate glasses leads to significant changes in the boron speciation, as 4-coordinated aluminum also requires charge stabilization, either through alkali cations or through formation of 5- and 6-fold coordinated aluminum. The introduction of Al₂O₃ in sodium borate glasses can lead to improved mechanical properties like hardness and, crack resistance. Al₂O₃ may also influence the structure of the glass and, additionally, lower the liquidus temperature and coefficient of thermal expansion, or enhance the strain point. In addition to its role as a network former, Al₂O₃(and ZrO₂) help improve the chemical durability in borate glass while having no toxicity concerns. In some embodiments, the glass can comprise 0-10 wt % Al₂O₃. In some embodiments, the glass can comprise 0-10 wt % Al₂O₃. In some embodiments, the glass can comprise from 0 to 10 wt %, 0 to 8 wt %, 0 to 6 wt %, 0 to 4 wt %, 0 to 2 wt %, >0 to 10 wt %, >0 to 8 wt %, >0 to 6 wt %, >0 to 4 wt %, >0 to 2 wt %, 1 to 10 wt %, 1 to 8 wt %, 1 to 6 wt %, 1 to 4 wt %, 1 to 2 wt %, 3 to 8 wt %, 3 to 6 wt %, 3 to 10 wt %, 5 to 8 wt %, 5 to 10 wt %, 7 to 10 wt %, or 8 to 10 wt % Al₂O₃. In some embodiments, the glass can comprise 0, >0, 1, 2, 3, 4, 5, 6, 7, 8, 9, or 10 wt % Al₂O₃.

P₂O₅ also serves as a network former. Furthermore, the liberation of phosphate ions to the surface of bioactive glasses contributes to the formation of apatite. The inclusion of phosphate ions in the bioactive glass increases apatite formation rate and the binding capacity of the bone tissue. In addition, P₂O₅ increases the viscosity of the glass, which in turn expands the range of operating temperatures, and is therefore an advantage to manufacture and formation of the glass. In some embodiments, the glass can comprise 50-90 wt % P₂O₅. In some embodiments, the glass can comprise 55-85 wt % P₂O₅. In some embodiments, the glass can comprise from 50-90 wt %, 55-90 wt %, 60-90 wt %, 65-90 wt %, 70-90 wt %, 75-90 wt %, 80-90 wt %, 50-85 wt %, 55-85 wt %, 60-85 wt %, 65-85 wt %, 70-85 wt %, 75-85 wt %, 80-85 wt %, 50-80 wt %, 55-80 wt %, 60-80 wt %, 65-80 wt %, 70-80 wt %, 75-80 wt %, 50-75 wt %, 55-75 wt %, 60-75 wt %, 65-75 wt %, 70-75 wt %, 50-70 wt %, 55-70 wt %, 60-70 wt %, 65-70 wt %, 50-65 wt %, 55-65 wt %, 60-65 wt %, 50-60 wt %, or 55-60 wt %, P₂O₅. In some embodiments, the glass can comprise about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 wt % P₂O₅.

Alkali oxides (Li₂O, Na₂O, K₂O, Rb₂O, or Cs₂O) serve as aids in achieving low melting temperature and low liquidus temperatures. Meanwhile, the addition of alkali oxides can improve bioactivity. Further, Na₂O and K₂O may influence the coefficient of thermal expansion, especially at low temperatures. In some embodiments, the glass can comprise from 0-15 wt % Na₂O. In some embodiments, the glass can comprise >0-10 wt % Na₂O. In some embodiments, the glass can comprise 2-8 wt % Na₂O. In some embodiments, the glass can comprise from 0-15 wt %, >0-15 wt %, 2-15 wt %, 5-15 wt %, 8-15 wt %, 10-15 wt %, 0-10 wt %, >0-10 wt %, 2-10 wt %, 5-10 wt %, 0-8 wt %, >0-8 wt %, 2-8 wt %, 5-8 wt %, 0-5 wt %, >0-5 wt %, or 2-5 wt % Na₂O. In some embodiments, the glass can comprise 0, >0, 1, 2, 3, 4, 5, 6, 7, 8, 9, 10, 11, 12, 13, 14, or 15 wt % Na₂O.

In some embodiments, the glass can comprise from 0-15 wt % K₂O. In some embodiments, the glass can comprise 2-8 wt % K₂O. In some embodiments, the glass can comprise 0-5 wt % K₂O. In some embodiments, the glass can comprise from 0-15 wt %, >0-15 wt %, 2-15 wt %, 5-15 wt %, 8-15 wt %, 10-15 wt %, 0-10 wt %, >0-10 wt %, 2-10 wt %, 5-10 wt %, 0-8 wt %, >0-8 wt %, 2-8 wt %, 5-8 wt %, 0-5 wt %, >0-5 wt %, or 2-5 wt % K₂O. In some embodiments, the glass can comprise 0, >0, 1, 2, 3, 4, 5, 6, 7, 8, 9, 10, 11, 12, 13, 14, or 15 wt % K₂O.

In some embodiments, the total amount of Na₂O and K₂O is important to the properties of the glass. In such embodiments, the glass can comprise a total of >6 wt % Na₂O and K₂O combined.

In some embodiments, Li₂O may be present and in such embodiments, the glass can comprise from 0-5 wt % Li₂O. In some embodiments, the glass can comprise from >0-5 wt % Li₂O. In some embodiments, the glass can comprise from about >0-3.5 wt % Li₂O. In some embodiments, the glass can comprise from 1-4 wt % Li₂O. In some embodiments, the glass can comprise from 0-5 wt %, 0-4 wt %, 0-3 wt %, 0-2 wt %, >0 to 5 wt %, >0 to 4 wt %, >0 to 3 wt %, >0 to 2 wt %, 1 to 5 wt %, 1 to 4 wt %, or 1 to 3 wt % Li₂O. In some embodiments, the glass can comprise about 0, >0, 1, 2, 3, 4, or 5 wt % Li₂O.

In some embodiments, the total amount of the alkalis Li₂O, Na₂O, and K₂O (R₂O) is important to the glass properties. In some embodiments, the glass can comprise 0-15 wt % R₂O, wherein R₂O is the sum or Li₂O, Na₂O, and K₂O. In some embodiments, the glass can comprise >0-10 wt % R₂O. In some embodiments, the glass can comprise 2-8 wt % R₂O. In some embodiments, the glass can comprise from 0-15 wt %, >0-15 wt %, 2-15 wt %, 5-15 wt %, 8-15 wt %, 10-15 wt %, 0-10 wt %, >0-10 wt %, 2-10 wt %, 5-10 wt %, 0-8 wt %, >0-8 wt %, 2-8 wt %, 5-8 wt %, 0-5 wt %, >0-5 wt %, or 2-5 wt % R₂O. In some embodiments, the glass can comprise 0, >0, 1, 2, 3, 4, 5, 6, 7, 8, 9, 10, 11, 12, 13, 14, or 15 wt % R₂O.

Divalent cation oxides (such as alkaline earth oxides) also improve the melting behavior and the bioactivity of the glass. Particularly, CaO is found to be able to react with P₂O₅ to form apatite when immersed in a simulated body fluid (SBF) or in vivo. The release of Ca²⁺ ions from the surface of the glass contributes to the formation of a layer rich in calcium phosphate.

In some embodiments, the glass can comprise 1-25 wt % CaO. In some embodiments, the glass can comprise 5-25 wt % CaO. In some embodiments, the glass can comprise 8 to 23 wt % R₂O. In some embodiments, the glass can comprise from 1-25, 1-23, 1-20, 1-15, 1-12, 1-10, 1-8, 3-25, 3-23, 3-20, 3-15, 3-12, 3-10, 3-8, 5-25, 5-23, 5-20, 5-15, 5-12, 3-10, 5-8, 8-25, 8-23, 8-20, 8-15, 8-12, 8-10, 10-25, 10-23, 10-20, 10-15, 15-25, 15-23, 15-20, or 20-25, wt % CaO. In some embodiments, the glass can comprise 1, 2, 3, 4, 5, 6, 7, 8, 9, 10, 11, 12, 13, 14, 15, 16, 17, 18, 19, 20, 21, 22, 23, 24, or 25 wt % CaO.

The combination of P₂O₅ and CaO may provide advantageous compositions for bioactive glasses. In some embodiments, the glass compositions comprise P₂O₅ and CaO with the sum of P₂O₅ and CaO being from 70 wt % or greater, 75 wt % or greater, 75-90 wt % or 80-90 wt %.

In some embodiments, the glasses comprise MgO. In some embodiments, the glass can comprise 0-10 wt % MgO. In some embodiments, the glass can comprise from 0 to 5 wt % MgO. In some embodiments, the glass can comprise from >0 to 10 wt %, 3 to 10 wt %, or 3 to 8 wt % MgO. In some embodiments, the glass can comprise from 0 to 10 wt %, 0 to 8 wt %, 0 to 6 wt %, 0 to 4 wt %, 0 to 2 wt %, >0 to 10 wt %, >0 to 8 wt %, >0 to 6 wt %, >0 to 4 wt %, >0 to 2 wt %, 1 to 10 wt %, 1 to 8 wt %, 1 to 6 wt %, 1 to 4 wt %, 1 to 2 wt %, 3 to 8 wt %, 3 to 6 wt %, 3 to 10 wt %, 5 to 8 wt %, 5 to 10 wt %, 7 to 10 wt %, or 8 to 10 wt % MgO. In some embodiments, the glass can comprise about 0, >0, 1, 2, 3, 4, 5, 6, 7, 8, 9, or 10 wt % MgO.

SrO may be present in some embodiments and in such embodiments, the glass can comprise from 0 to 10 wt % SrO. In some embodiments, the glass can comprise from >0 to 10 wt % SrO. In some embodiments, the glass can comprise from 3 to 10 wt %, 5 to 10 wt %, 5 to 8 wt % SrO. In some embodiments, the glass can comprise from 0 to 10 wt %, 0 to 8 wt %, 0 to 6 wt %, 0 to 4 wt %, 0 to 2 wt %, >0 to 10 wt %, >0 to 8 wt %, >0 to 6 wt %, >0 to 4 wt %, >0 to 2 wt %, 1 to 10 wt %, 1 to 8 wt %, 1 to 6 wt %, 1 to 4 wt %, 1 to 2 wt %, 3 to 8 wt %, 3 to 6 wt %, 3 to 10 wt %, 5 to 8 wt %, 5 to 10 wt %, 7 to 10 wt %, or 8 to 10 wt % SrO. In some embodiments, the glass can comprise about >0, 1, 2, 3, 4, 5, 6, 7, 8, 9, or 10 wt % SrO.

BaO may be present in some embodiments and in such embodiments, the glass can comprise from 0 to 15 wt % BaO. In some embodiments, the glass can comprise from 0 to 10 wt %, >0 to 5 wt %, 6 to 13 wt %, 5 to 15 wt %, 7 to 13 wt %, 7 to 11 wt %, 8 to 12 wt % BaO. In some embodiments, the glass can comprise from 0 to 15 wt %, 0 to 13 wt %, 0 to 11 wt %, 0 to 9 wt %, 0 to 7 wt %, 0 to 5 wt %, >0 to 15 wt %, >0 to 13 wt %, >0 to 11 wt %, >0 to 9 wt %, >0 to 7 wt %, >0 to 5 wt %, 1 to 15 wt %, 1 to 13 wt %, 1 to 11 wt %, 1 to 9 wt %, 1 to 7 wt %, 1 to 5 wt %, 3 to 15 wt %, 3 to 13 wt %, 3 to 11 wt %, 3 to 9 wt %, 3 to 7 wt %, 3 to 5 wt %, 5 to 15 wt %, 5 to 13 wt %, 5 to 11 wt %, 5 to 9 wt %, 5 to 7 wt %, 7 to 15 wt %, 7 to 13 wt %, 7 to 11 wt %, 7 to 9 wt %, 9 to 15 wt %, 9 to 13 wt %, 9 to 11 wt %, 11 to 15 wt %, or 11 to 13 wt % BaO. In some embodiments, the glass can comprise about 0, >0, 1, 2, 3, 4, 5, 6, 7, 8, 9, 10, 11, 12, 13, 14, or 15 wt % BaO.

Alkaline earth oxides may improve other desirable properties in the materials, including influencing the Young's modulus and the coefficient of thermal expansion. In some embodiments, the glass comprises from 5-30 wt % MO (5 wt %<MO<30 wt %), where M is the sum of the alkaline earth metals Mg, Ca, Sr, and Ba, in the glass. In some embodiments, the glass can comprise from 5 to 25 wt % MO. In some embodiments, the glass can comprise about 5, 6, 7, 8, 9, 10, 11, 12, 13, 14, 15, 16, 17, 18, 19, 20, 21, 22, 23, 24, 25, 26, 27, 28, 29, or 30 wt % MO.

In some embodiments, the glasses comprise ZnO. In some embodiments, the glass can comprise 0-10 wt % ZnO. In some embodiments, the glass can comprise from 0 to 5 wt % ZnO. In some embodiments, the glass can comprise from >0 to 10 wt %, 3 to 10 wt %, or 3 to 8 wt % ZnO. In some embodiments, the glass can comprise from 0 to 10 wt %, 0 to 8 wt %, 0 to 6 wt %, 0 to 4 wt %, 0 to 2 wt %, >0 to 10 wt %, >0 to 8 wt %, >0 to 6 wt %, >0 to 4 wt %, >0 to 2 wt %, 1 to 10 wt %, 1 to 8 wt %, 1 to 6 wt %, 1 to 4 wt %, 1 to 2 wt %, 3 to 8 wt %, 3 to 6 wt %, 3 to 10 wt %, 5 to 8 wt %, 5 to 10 wt %, 7 to 10 wt %, or 8 to 10 wt % ZnO. In some embodiments, the glass can comprise about 0, >0, 1, 2, 3, 4, 5, 6, 7, 8, 9, or 10 wt % ZnO.

Additional components can be incorporated into the glass to provide additional benefits or may be incorporated as contaminants typically found in commercially-prepared glass. For example, additional components can be added as coloring or fining agents (e.g., to facilitate removal of gaseous inclusions from melted batch materials used to produce the glass) and/or for other purposes. In some embodiments, the glass may comprise one or more compounds useful as ultraviolet radiation absorbers. In some embodiments, the glass can comprise 3 wt % or less ZnO, TiO₂, CeO, MnO, Nb₂O₅, MoO₃, Ta₂O₅, WO₃, SnO₂, Fe₂O₃, As₂O₃, Sb₂O₃, Cl, Br, or combinations thereof. In some embodiments, the glass can comprise from 0 to about 3 wt %, 0 to about 2 wt %, 0 to about 1 wt %, 0 to 0.5 wt %, 0 to 0.1 wt %, 0 to 0.05 wt %, or 0 to 0.01 wt % ZnO, TiO₂, CeO, MnO, Nb₂O₅, MoO₃, Ta₂O₅, WO₃, SnO₂, Fe₂O₃, As₂O₃, Sb₂O₃, Cl, Br, or combinations thereof. The glasses, according to some embodiments, can also include various contaminants associated with batch materials and/or introduced into the glass by the melting, fining, and/or forming equipment used to produce the glass. For example, in some embodiments, the glass can comprise from 0 to about 3 wt %, 0 to about 2 wt %, 0 to about 1 wt %, 0 to about 0.5 wt %, 0 to about 0.1 wt %, 0 to about 0.05 wt %, or 0 to about 0.01 wt % SnO₂ or Fe₂O₃, or combinations thereof.

Non-limiting examples of amounts of precursor oxides for forming the embodied glasses are listed in Table 1, along with the properties of the resulting glasses.

TABLE 1

| Oxide | | | | | | | | | |
|---|---|---|---|---|---|---|---|---|---|
| (wt %) | 1 | 2 | 3 | 4 | 5 | 6 | 7 | 8 | 9 |
| SiO₂ | 0.1 | 0.1 | 0.0 | 0.0 | 0.0 | 0.0 | 0.0 | 0.0 | 0.0 |
| B₂O₃ | 3.4 | 7.1 | 13.0 | 4.6 | 11.4 | 0.0 | 4.9 | 9.0 | 0.0 |
| P₂O₅ | 63.2 | 64.0 | 68.2 | 78.0 | 76.4 | 73.5 | 66.5 | 61.0 | 77.3 |
| Al₂O₃ | 5.4 | 5.2 | 4.9 | 4.5 | 0.0 | 0.0 | 0.0 | 0.0 | 0.0 |
| Li₂O | 0.0 | 0.0 | 0.0 | 0.0 | 0.0 | 0.0 | 0.0 | 0.0 | 0.0 |
| Na₂O | 6.7 | 6.4 | 5.9 | 5.5 | 0.0 | 3.2 | 3.4 | 3.6 | 3.8 |
| K₂O | 0.0 | 0.0 | 0.0 | 0.0 | 0.0 | 4.5 | 4.8 | 5.2 | 5.5 |
| MgO | 0.0 | 0.0 | 0.0 | 0.0 | 0.0 | 1.1 | 1.2 | 1.2 | 1.3 |
| CaO | 21.2 | 17.2 | 8.0 | 7.4 | 12.1 | 15.9 | 17.2 | 17.9 | 9.8 |
| ZnO | 0.0 | 0.0 | 0.0 | 0.0 | 0.0 | 1.8 | 2.0 | 2.0 | 2.2 |
| ZrO₂ | 0.0 | 0.0 | 0.0 | 0.0 | 0.0 | 0.0 | 0.0 | 0.0 | 0.0 |
| Oxide | | | | | | | | | |
| (wt %) | 10 | 11 | 12 | 13 | 14 | 15 | 16 | 17 | 18 |
| SiO₂ | 0.0 | 0.0 | 0.0 | 0.0 | 0.0 | 0.0 | 0.0 | 0.0 | 0.0 |
| B₂O₃ | 0.8 | 0.6 | 0.6 | 2.6 | 5.4 | 0.0 | 0.6 | 0.6 | 0.8 |
| P₂O₅ | 82.7 | 81.4 | 77.6 | 69.6 | 59.8 | 77.3 | 77.6 | 81.4 | 82.7 |
| Al₂O₃ | 0.0 | 0.0 | 0.0 | 0.0 | 0.0 | 0.0 | 0.0 | 0.0 | 0.0 |
| Li₂O | 0.0 | 0.0 | 0.0 | 0.0 | 0.0 | 0.0 | 0.0 | 0.0 | 0.0 |
| Na₂O | 3.5 | 3.0 | 2.9 | 3.4 | 3.6 | 3.8 | 2.9 | 3.0 | 3.6 |
| K₂O | 5.1 | 4.2 | 4.1 | 4.8 | 5.2 | 5.5 | 4.1 | 4.2 | 5.1 |
| MgO | 1.2 | 1.1 | 1.1 | 1.1 | 1.3 | 1.3 | 1.1 | 1.1 | 1.2 |
| CaO | 4.6 | 7.9 | 11.8 | 16.5 | 22.6 | 9.8 | 11.9 | 7.8 | 4.6 |
| ZnO | 2.1 | 1.8 | 1.8 | 1.9 | 2.0 | 2.2 | 1.8 | 1.8 | 2.1 |
| ZrO₂ | 0.0 | 0.0 | 0.0 | 0.0 | 0.0 | 0.0 | 0.0 | 0.0 | 0.0 |

The glass compositions disclosed herein can be in any form that is useful for the medical and dental processes disclosed. The compositions can be in the form of, for example, particles, powder, microspheres, fibers, sheets, beads, scaffolds, woven fibers.

### Processes for Making Glasses

Glasses having the oxide contents listed in Table 1 can be made via traditional methods. For example, in some embodiments, the precursor glasses can be formed by thoroughly mixing the requisite batch materials (for example, using a turbular mixer) in order to secure a homogeneous melt, and subsequently placing into silica and/or platinum crucibles. The crucibles can be placed into a furnace and the glass batch melted and maintained at temperatures ranging from 1250−1650° C. for times ranging from about 6-16 hours. The melts can thereafter be poured into steel molds to yield glass slabs. Subsequently, those slabs can be transferred immediately to an annealer operating at about 500-650° C., where the glass is held at temperature for about 1 hour and subsequently cooled overnight. In another non-limiting example, precursor glasses are prepared by dry blending the appropriate oxides and mineral sources for a time sufficient to thoroughly mix the ingredients. The glasses are melted in platinum crucibles at temperatures ranging from about 1100° C. to about 1650° C. and held at temperature for about 16 hours. The resulting glass melts are then poured onto a steel table to cool. The precursor glasses are then annealed at appropriate temperatures.

The embodied glass compositions can be ground into fine particles in the range of 1-10 microns (μm) by air jet milling or short fibers. The particle size can be varied in the range of 1-100 μm using attrition milling or ball milling of glass frits. Furthermore, these glasses can be processed into short fibers, beads, sheets or three-dimensional scaffolds using different methods. Short fibers are made by melt spinning or electric spinning; beads can be produced by flowing glass particles through a hot vertical furnace or a flame torch; sheets can be manufactured using thin rolling, float or fusion-draw processes; and scaffolds can be produced using rapid prototyping, polymer foam replication and particle sintering. Glasses of desired forms can be used to support cell growth, soft and hard tissue regeneration, stimulation of gene expression or angiogenesis.

Continuous fibers can be easily drawn from the claimed composition using processes known in the art. For example, fibers can be formed using a directly heated (electricity passing directly through) platinum bushing. Glass cullet is loaded into the bushing, heated up until the glass can melt. Temperatures are set to achieve a desired glass viscosity (usually <1000 poise) allowing a drip to form on the orifice in the bushing (Bushing size is selected to create a restriction that influences possible fiber diameter ranges). The drip is pulled by hand to begin forming a fiber. Once a fiber is established it is connected to a rotating pulling/collection drum to continue the pulling process at a consistent speed. Using the drum speed (or revolutions per minute RPM) and glass viscosity the fiber diameter can be manipulated—in general the faster the pull speed, the smaller the fiber diameter. Glass fibers with diameters in the range of 1-100 μm can be drawn continuously from a glass melt (FIG. 4). Fibers can also be created using an updraw process. In this process, fibers are pulled from a glass melt surface sitting in a box furnace. By controlling the viscosity of the glass, a quartz rod is used to pull glass from the melt surface to form a fiber. The fiber can be continuously pulled upward to increase the fiber length. The velocity that the rod is pulled up determines the fiber thickness along with the viscosity of the glass.

### Glass Bioactivity

Aspects are related to compositions or matrices containing embodied bioactive glass compositions and the methods of using the matrices to treat medical conditions. The matrices can be a toothpaste, mouthwash, rinse, spray, ointment, salve, cream, bandage, polymer film, oral formulation, pill, capsule, transdermal formulation, and the like. The bioactive glass compositions claimed can be physically or chemically attached to matrices or other matrix components, or simply mixed in. As noted above, the bioactive glass can be in any form that works in the application, including particles, beads, particulates, short fibers, long fibers, or woolen meshes. The methods of using the glass-containing matrices to treat a medical condition can be simply like the use of matrix as normally applied.

A. Oral Health

Example compositions exhibit a continuous calcium release, which has been well recognized to be critical for treating dentin hypersensitivity, tooth remineralization and soft tissue regeneration. As a result of calcium ion release, the embodied compositions can react with saliva to form hydroxycarbonated apatite (HCA) or fluorapatite, exhibiting tubule occlusion at the surface by the formation of a smear layer and within dentin tubules, and rebuild, strengthen, and protect tooth structure. Fluoride may be incorporated into the glass compositions in the precursor form of sodium fluoride (NaF), stannous fluoride (SnF₂), or calcium fluoride (CaF₂). In oral fluid the fluoride-incorporating glasses will release fluoride and form fluorapatite, which is more resistant to acid dissolution than hydroxycarbonated apatite.

In an additional embodiment, glass can be formulated in a non-aqueous dentifrice product. A typical non-aqueous toothpaste formulation is shown in Table 2.

TABLE 2
Exemplary Toothpaste compositions in weight percent

| | Toothpaste | Toothpaste |
|---|---|---|
| Ingredients | Base | Base + Bioglass |
| Glycerol | 69.0 | 64.0 |
| Sodium lauryl sulfate | 1.1 | 1.1 |
| PEG400 | 20 | 20 |
| Silicon dioxide | 8 | 8 |
| Bioactive glass | 0 | 5 |
| Carbopol 940 | 0.5 | 0.5 |
| Sodium saccharin | 0.35 | 0.35 |
| Flavor | 1.03 | 1.03 |
| Total | 100 | 100 |

B. Antimicrobial/Wound Healing Applications

In the United States, non-healing wounds affect 3 to 6 million people with 85% of them being persons 65 years old. Each year, the total cost for the health care expenditures for non-healing wounds is estimated to be more than $3 billon. These non-healing wounds frequently turn into a state of pathologic inflammation due to a postponed, incomplete, or uncoordinated healing process. And most of them are ulcers associated with ischemia, diabetes mellitus, venous stasis disease or stress. There is continuing need for new approaches and methods to accelerating the healing process for wounds including lacerations, diabetic ulcers, bed sores, burns and so on.

Wound healing is a dynamic process and is achieved through four continuous phases: rapid hemostasis, appropriate inflammation, proliferation, and remodeling. Many factors can influence the wound healing process. Local factors are mainly oxygen supply and infections via microorganisms while systemic factors including age, stress, sex hormones, diseases, nutrition and so on. The embodied bioactive glass compositions disclosed herein support one or more of these phases and can act via fast ion release, prevent bacterial growth, or promote endothelial cell migration and be easily formed into different forming factors for wound dressing or covering.

In embodiments, the composition can exhibit a continuous calcium release, which has been well recognized to be critical for treating soft tissue regeneration. The release of ions such as Na⁺ and Ca²⁺ is believed to account for the improved in vitro performance of the example composition. As a result of ion release, a fast endothelial cell migration may be observed when cultured in a glass-extraction-containing medium, making the embodied bioactive glass compositions useful for skin repair, wound healing, tissue engineering, and cosmetic applications.

Furthermore, some embodied compositions may demonstrate antibacterial capabilities. Testing of composition would show the ability of the example compositions to prevent bacteria growth, which is important for both wound healing and oral health.

While typical embodiments have been set forth for the purpose of illustration, the foregoing description should not be deemed to be a limitation on the scope of the disclosure or appended claims. Accordingly, various modifications, adaptations, and alternatives may occur to one skilled in the art without departing from the spirit and scope of the present disclosure or appended claims.

1. A glass composition comprising, wt %:
0-25 SiO₂
0-15 B₂O₃
50-90 P₂O₅
0-10 Al₂O₃
0-5 Li₂O
0-15 Na₂O
0-15 K₂O
0-10 MgO
1-25 CaO
5-30 MO
0-15 R₂O, and
70 wt % or greater (P₂O₅+CaO)
wherein MO is the sum of MgO, CaO, SrO, and BaO, R₂O is the sum of Na₂O, K₂O,
Li₂O, and Rb₂O.

2. The glass composition of claim 1, wherein the glass composition comprises:
>0-10 wt % Na₂O.

3. The composition of claim 1, wherein the glass composition comprises:
2-8 wt % Na₂O.

4. The composition of of claim 1, wherein the glass composition comprises:
>0-15 wt % B₂O₃.

5. The glass composition of claim 1, wherein the glass composition comprises:
75 wt % or greater (P₂O₅+CaO).

6. The glass composition of claim 5, wherein the glass composition comprises:
80 wt % or greater (P₂O₅+CaO).

7. The glass composition of claim 1, wherein the glass composition comprises:
>0-5 wt % MgO.

8. The glass composition of claim 1, wherein the glass composition comprises:
>0-8 wt % ZnO₂.

9. The glass composition of claim 1, wherein the glass composition comprises:
59-70 wt % P₂O₅;
2-15 wt % B₂O₃; and
5-25 wt % CaO.

10. The glass composition of claim 1, wherein the glass composition comprises:
70-80 wt % P₂O₅;
9-15 wt % CaO; and
is essential free of Al₂O₃.

11. The glass composition of claim 1, wherein the glass composition comprises:
70-80 wt % P₂O₅;
5-15 wt % CaO; and
1-5 wt % Na₂O.

12. The glass composition of claim 1, wherein the glass composition further comprises:
0-5 wt % ZrO₂.

13. The glass composition of claim 1, wherein the glass composition is essentially free of or comprises 1 wt % or less of Li₂O.

14. The glass composition of claim 1, wherein the glass composition is essentially free of or comprises 1 wt % or less of SiO₂.

15. The glass composition of claim 1, wherein the glass composition is in the form of powder, particles, beads, particulates, short fibers, long fibers, or woolen meshes.

16. A method of making the glass composition of of claim 1, the method comprising:
a. mixing the requisite batch oxides to form a mixture; and
b. extracting the mixture to form a glass comprising the composition.

17. The method of claim 16, wherein the glass is milled to form a plurality of particles, the particles having a particle distribution that is approximately Gaussian.

18. The method of claim 17, wherein the glass composition is in the form of a plurality of particles, and the particles have an average particle size of from 10 microns to 100 microns.

19. An oral care composition comprising the glass composition of claim 1.

20. The oral care composition of claim 19, wherein the glass composition is in the form of a plurality of particles, the particles having a particle distribution that is approximately Gaussian.

21-27. (canceled)
